(19) United States
(12) Patent Application Publication
(10) Pub. No.: US 2017/0325786 A1
(43) Pub. Date: Nov. 16, 2017

(54) MOBILE ULTRASOUND

(71) Applicant: Brian Pepe, Newton, PA (US)

(72) Inventor: Brian Pepe, Newton, PA (US)

(21) Appl. No.: 15/592,191

(22) Filed: May 10, 2017

Related U.S. Application Data
(60) Provisional application No. 62/334,323, filed on May 10, 2016.

(51) Int. Cl. A61B 8/00; G06Q 10/10; G06F 19/00; G06T 3/40

(52) U.S. Cl. CPC A61B 8/565; G06T 3/40; G06Q 10/1095; G06F 19/3425; A61B 8/4483; G06F 19/322; G06F 19/321; G06F 19/327

(57) ABSTRACT

Ultrasound images are captured and streamed to a consulting physician and reviewed by the physician in real time. In an embodiment, output of an ultrasound machine is sent to a transcoder, which sends the images to a livestreaming service. A live stream of the images is then sent to the physician, who may view the images in real time and provide feedback in real time is desired.

### CROSS-REFERENCE TO RELATED APPLICATIONS

This application claims priority benefit of U.S. Provisional Patent Application No. 62/334,323 (Docket # CN-1), entitled “MOBILE ULTRASOUND,” filed May 10, 2016, by Brian Pepe, which is incorporated herein by reference.

### FIELD

This specification is related to providing medical images or other examination information to a consulting doctor.

### BACKGROUND

The subject matter discussed in the background section should not be assumed to be prior art merely as a result of its mention in the background section. Similarly, a problem mentioned in the background section or associated with the subject matter of the background section should not be assumed to have been previously recognized in the prior art. The subject matter in the background section merely represents different approaches, which in and of themselves may also be inventions.

Ultrasound imaging is well known. However, the physician may not necessarily be able to be present while the ultrasound image is being taken. Yet, the patient may need or desire an immediate diagnosis and/or other immediate feedback regarding images taken. Accomplishing tele-ultrasound in real time in a manner that is easy to work with and understand (as opposed to being complex and convoluted) is nontrivial.

### BRIEF DESCRIPTION

In the following drawings like reference numbers are used to refer to like elements. Although the following figures depict various examples of the invention, the invention is not limited to the examples depicted in the figures.

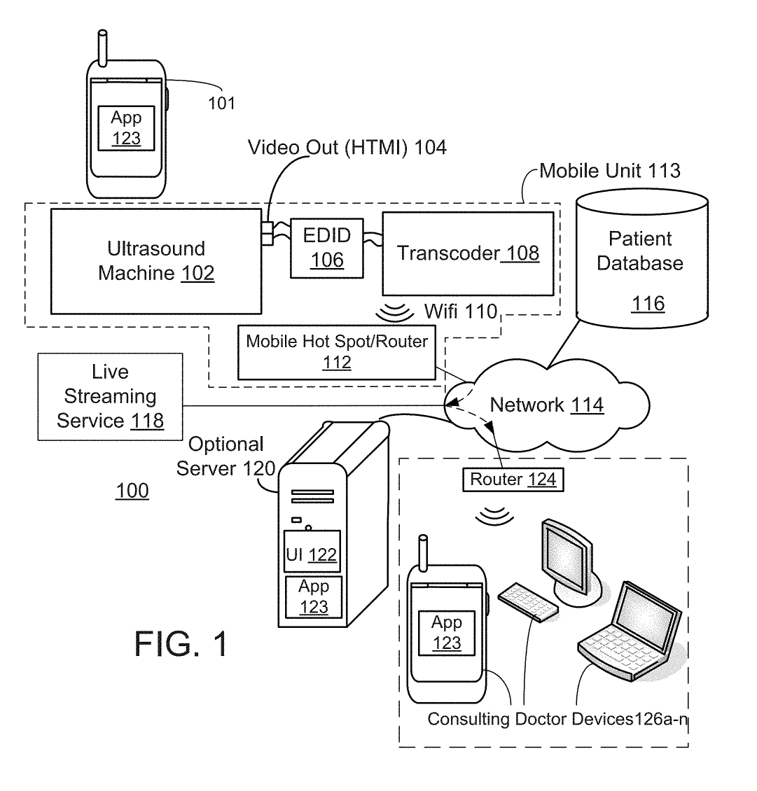

FIG. 1 shows a block diagram of an embodiment of a mobile ultrasound system.

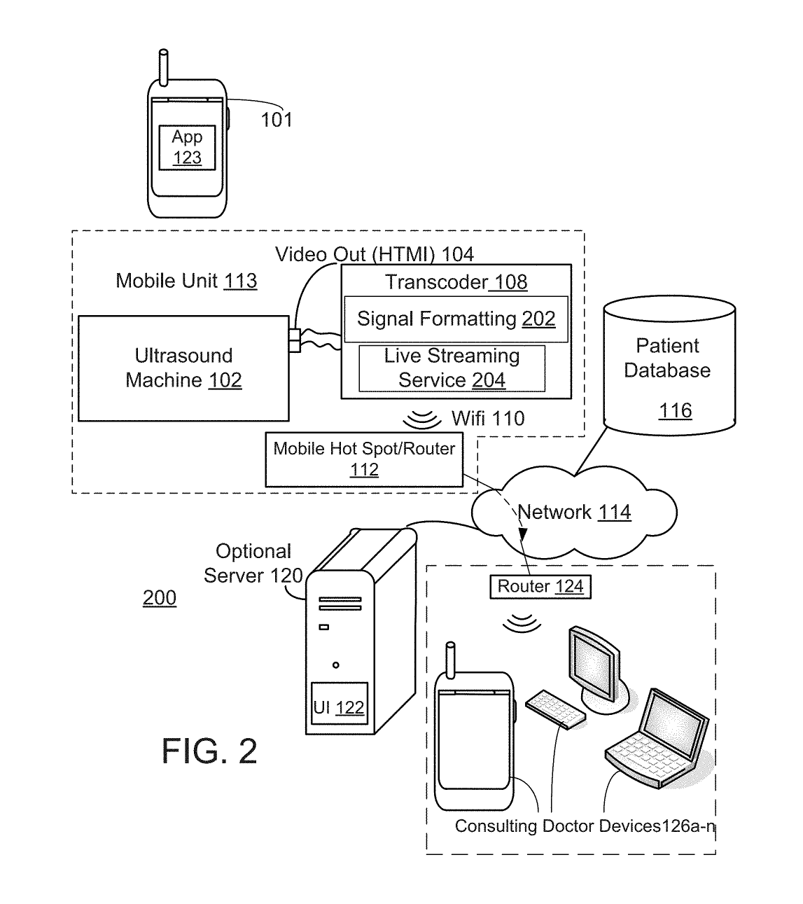

FIG. 2 shows a block diagram of another embodiment of a mobile ultrasound system.

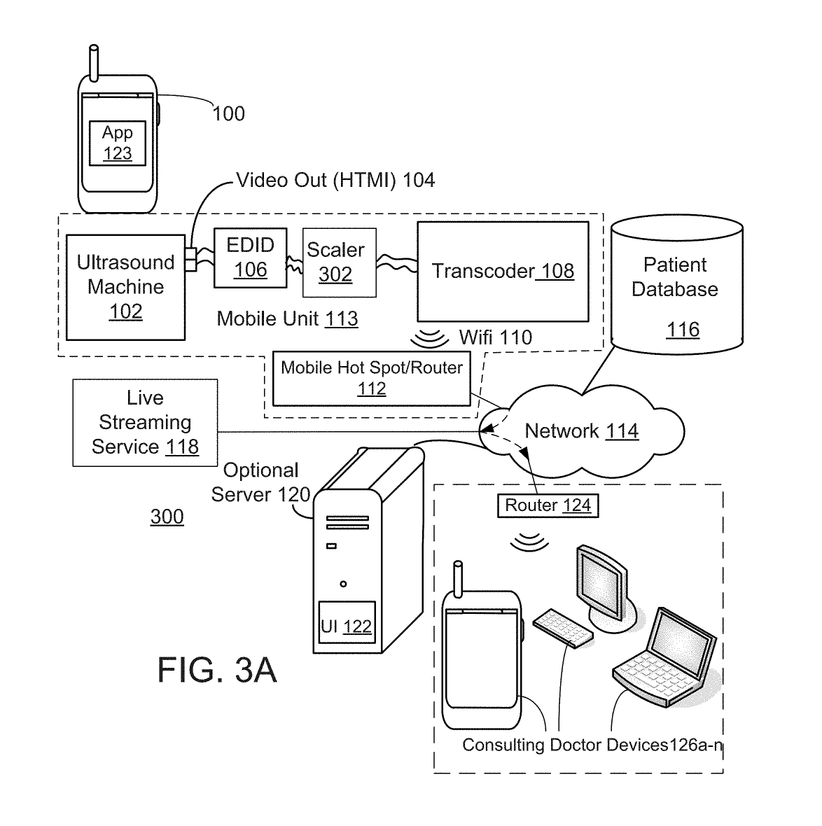

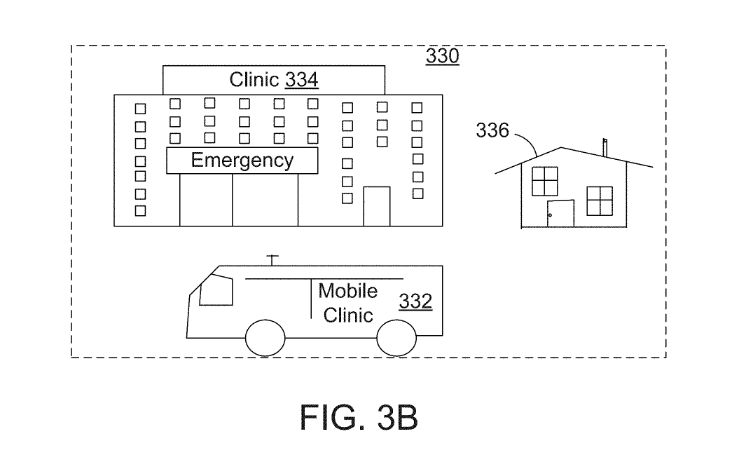

FIG. 3A shows a block diagram of another embodiment of a mobile ultrasound system.

FIG. 3B shows a representation of an embodiment of an environment in which the ultrasound system of FIGS. 1-3A may be used.

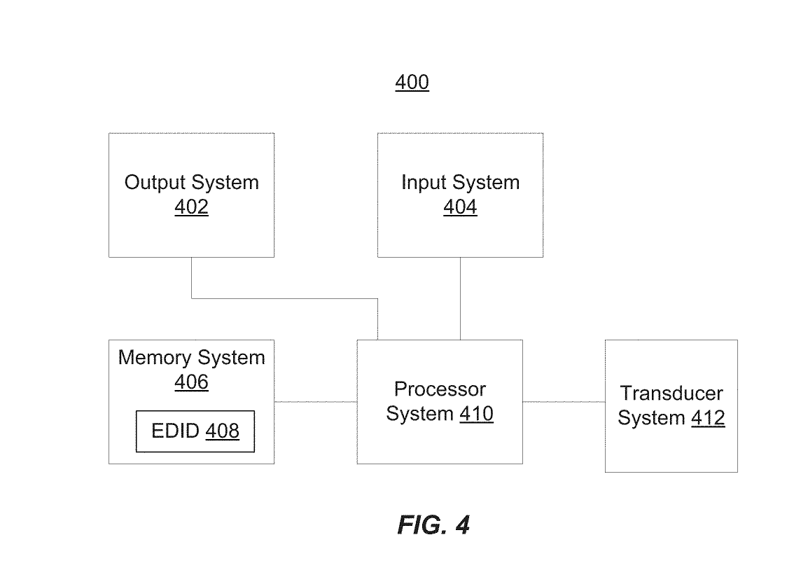

FIG. 4 shows a block diagram of an embodiment of an ultrasound system used in the system of FIGS. 1-3A.

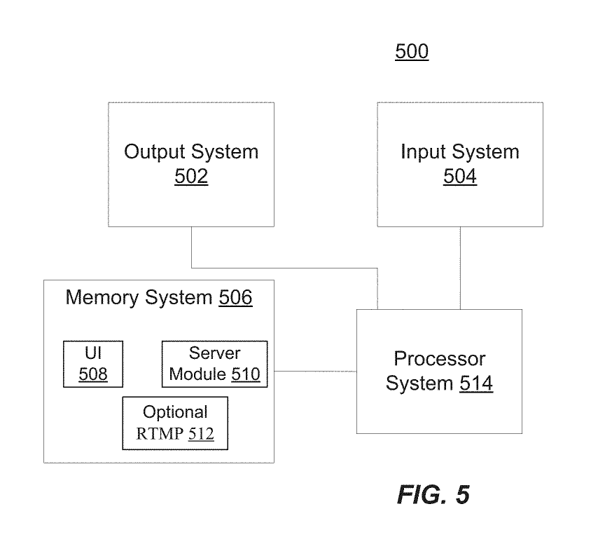

FIG. 5 shows a block diagram of an embodiment of a server system used in the system of FIG. 1, 2, or 3A.

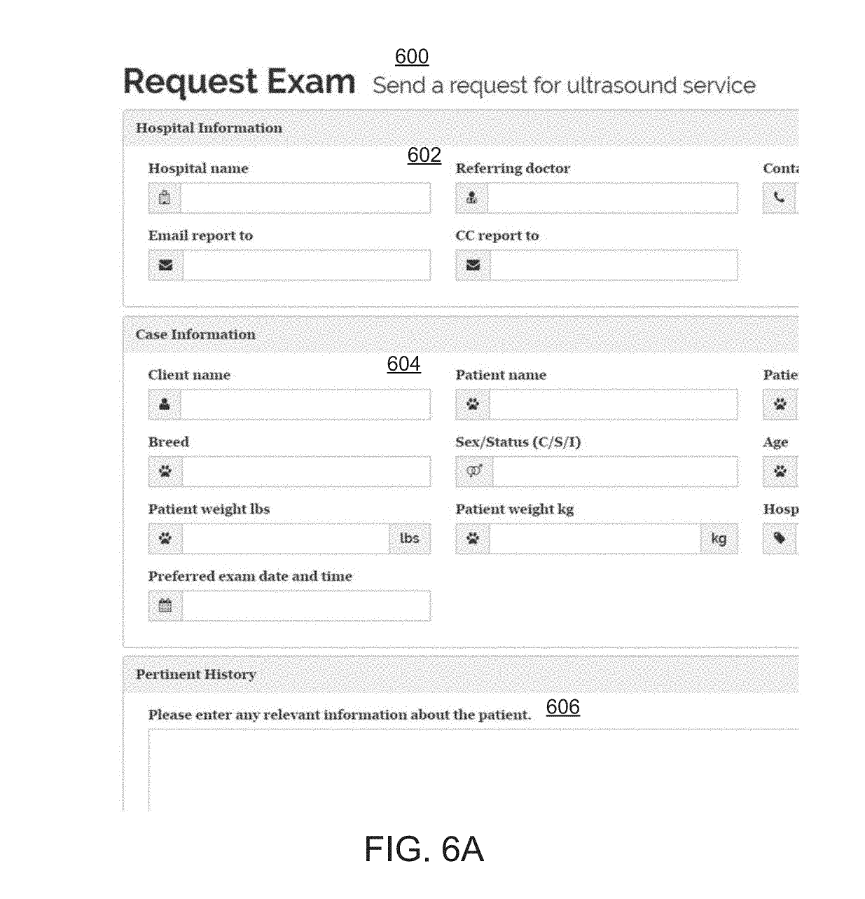

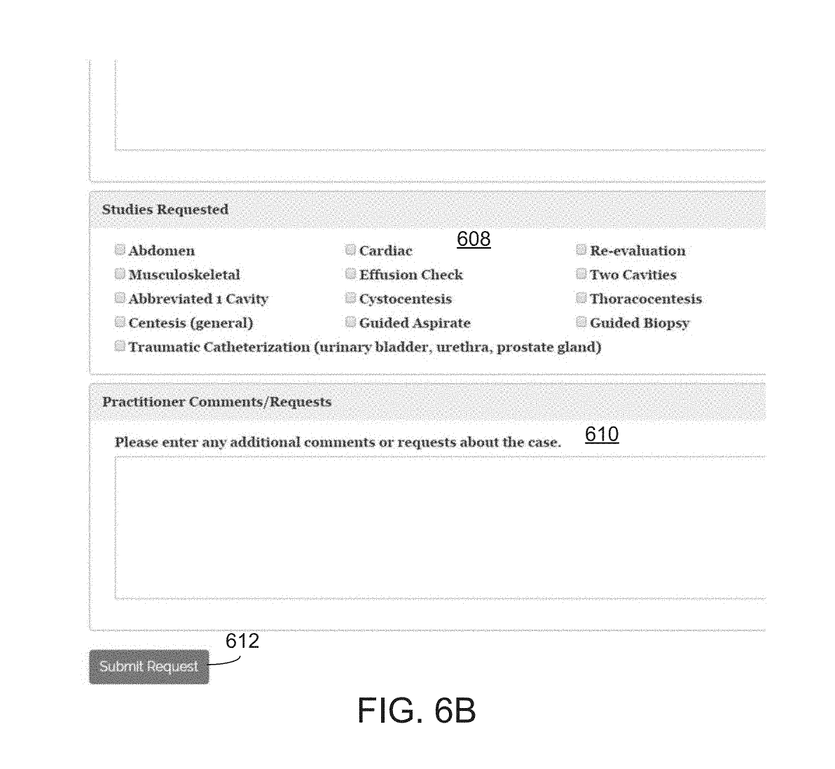

FIGS. 6A and 6B shows an example of a form for a request for an ultrasound examination.

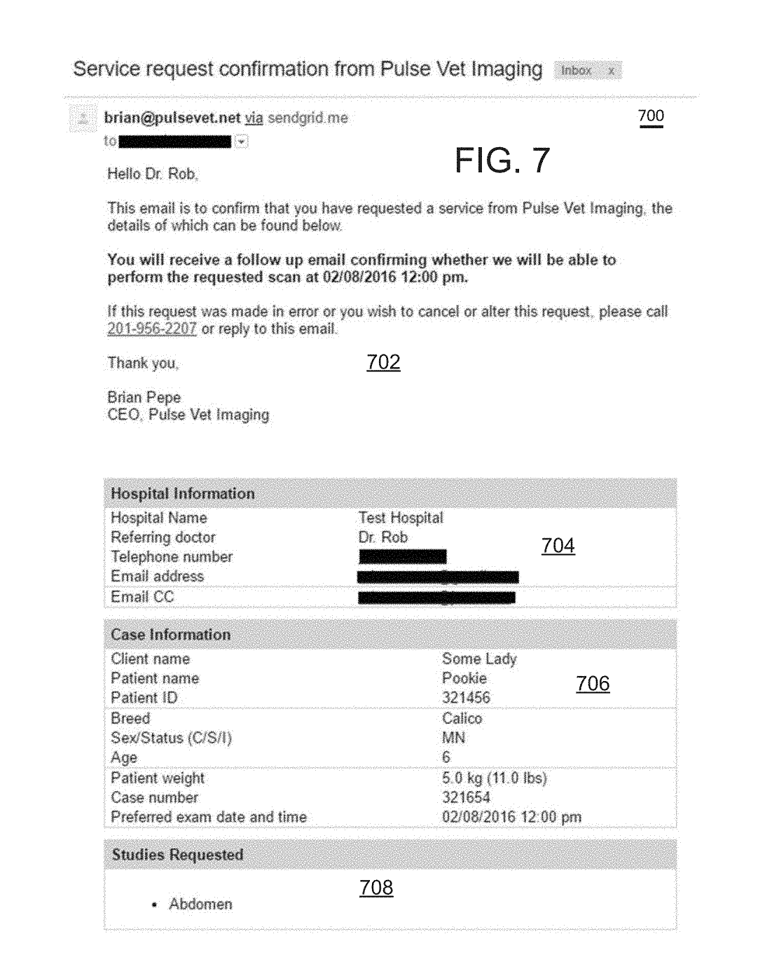

FIG. 7 shows a screenshot of an example of a page acknowledging a request for an ultrasound examination (e.g., a request made, via the form of FIGS. 6A and 6B).

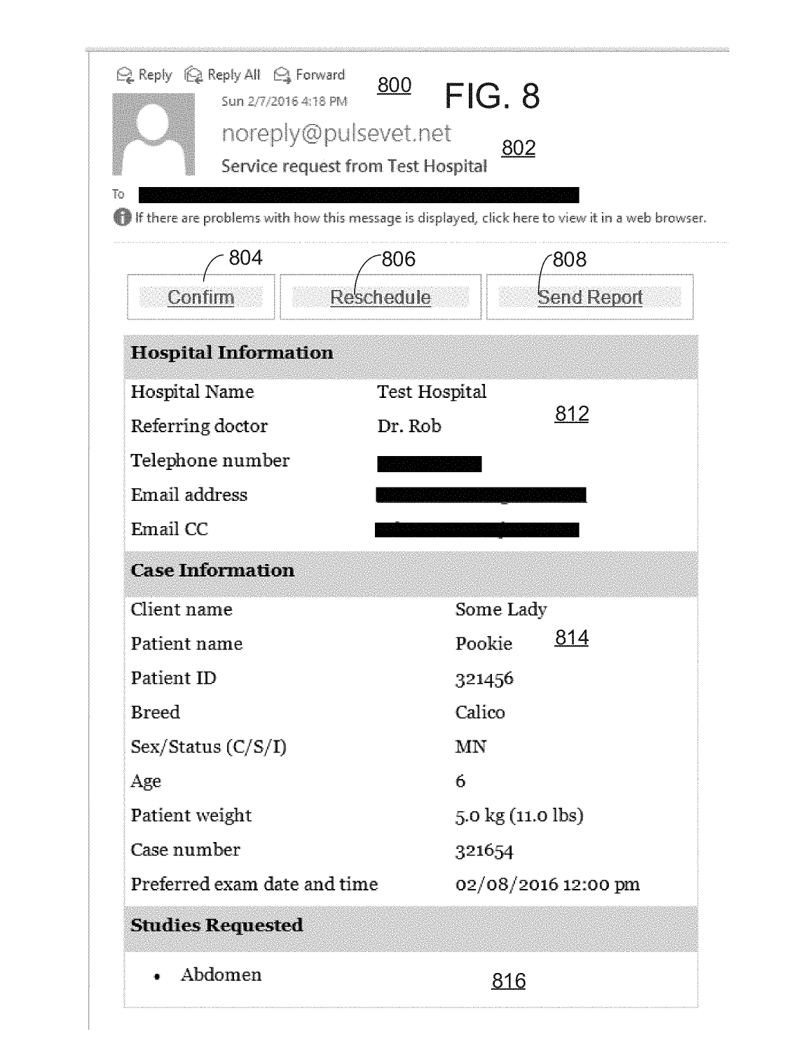

FIG. 8 shows a screenshot of an example of a page that the ultrasound practitioner may use to respond to the request for an appointment for performing an ultrasound examination.

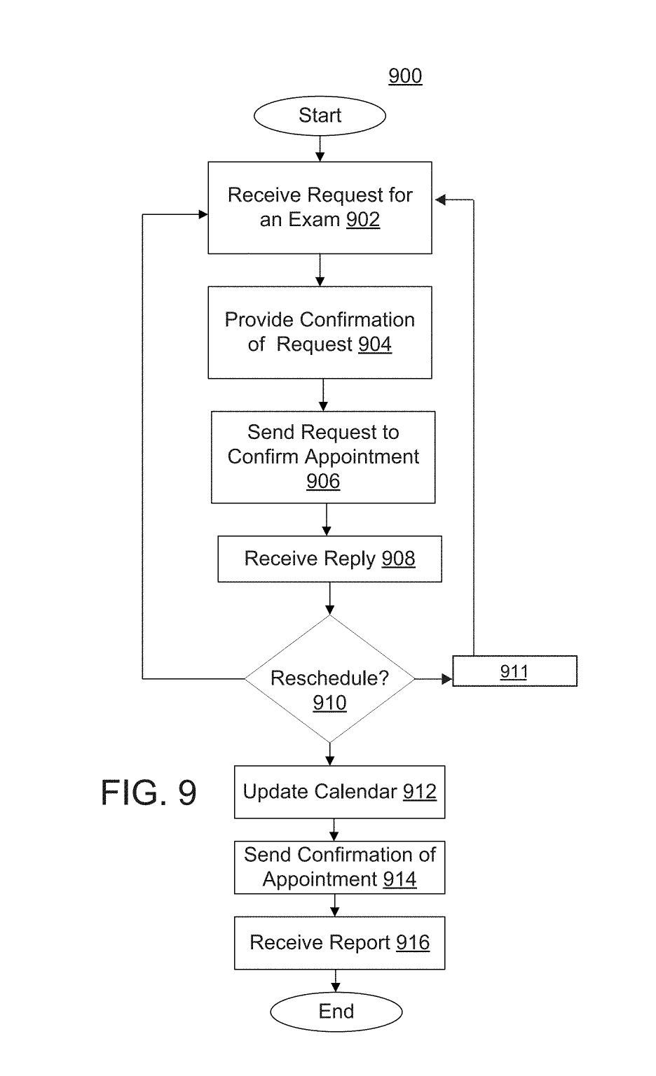

FIG. 9 is an embodiment of a method of scheduling and reporting an ultrasound examination, which is from the point of view of the server.

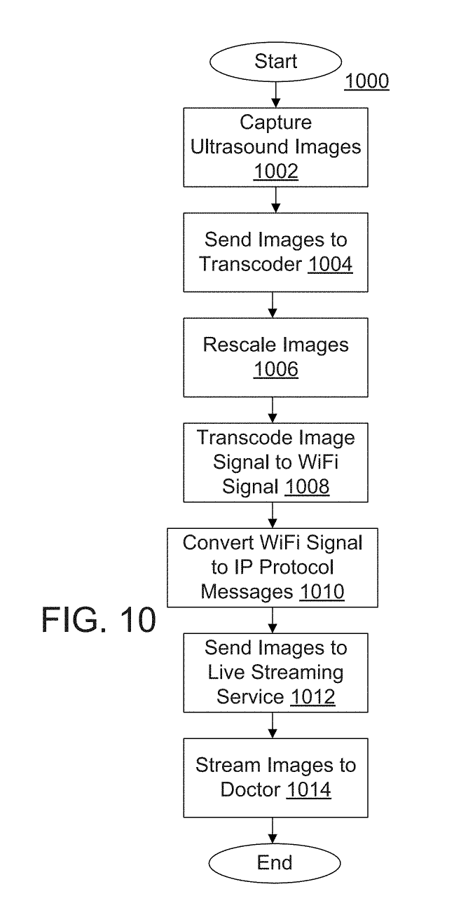

FIG. 10 is an embodiment of a method performing an ultrasound examination.

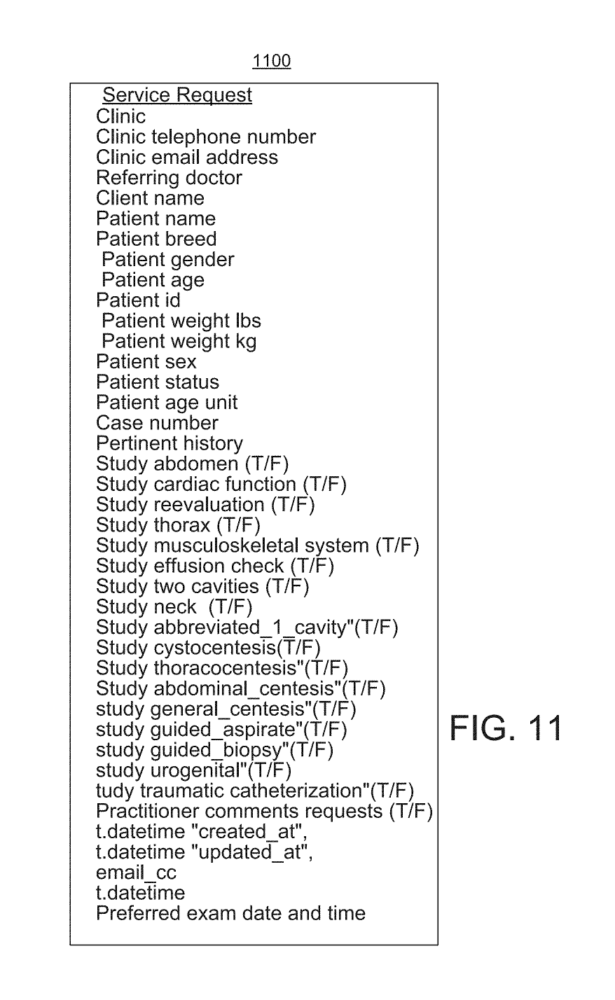

FIG. 11 illustrates an example of a first embodiment of a schema for the system of FIG. 1, 2, or 3A.

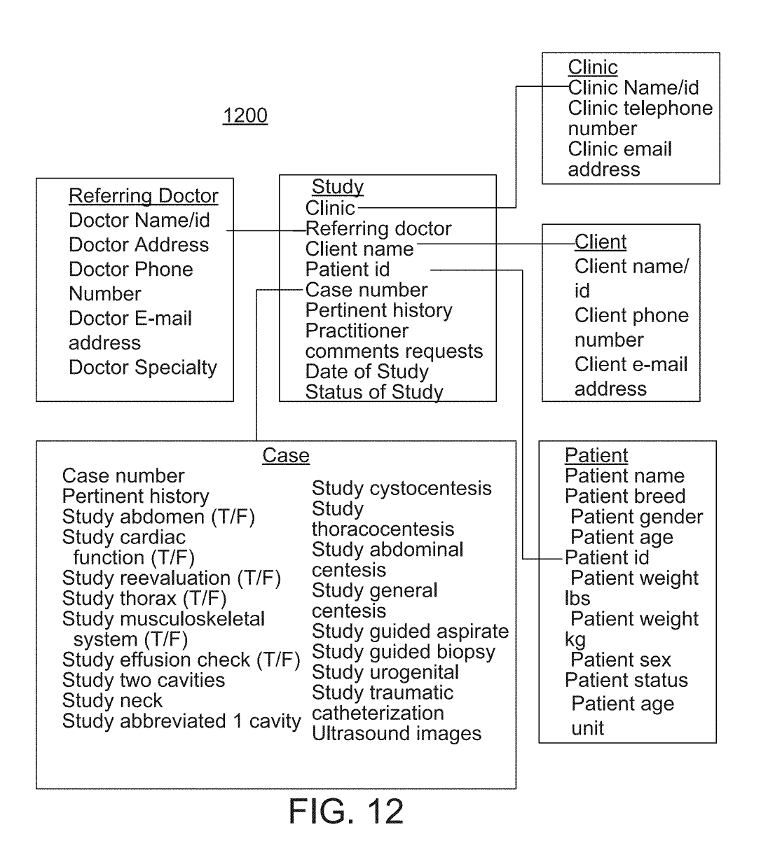

FIG. 12 is another embodiment of a schema for the system of FIG. 1, 2, or 3A.

### DETAILED DESCRIPTION

Although various embodiments of the invention may have been motivated by various deficiencies with the prior art, which may be discussed or alluded to in one or more places in the specification, the embodiments of the invention do not necessarily address any of these deficiencies. In other words, different embodiments of the invention may address different deficiencies that may be discussed in the specification. Some embodiments may only partially address some deficiencies or just one deficiency that may be discussed in the specification, and some embodiments may not address any of these deficiencies.

In an embodiment, results of live ultrasound examinations are streamed to a specialist who interprets the examination and returns an immediate result, such as a diagnosis.

### Real Time Mobile Ultrasound

FIG. 1 shows a block diagram of an embodiment of an ultrasound system 100. In an embodiment, ultrasound system 100 includes communication device 101, ultrasound machine 102, video out 104, EDID 106, transcoder 108, WiFi 110, mobile hotspot/router 112, mobile unit 113, network 114, patient database 116, live streaming service 118, optional server 120, UI 122, App 123, router 124, doctor devices 126a-n. In other embodiments ultrasound system 100 may not have all of the elements or features listed and/or may have other elements or features instead of or in addition to those listed.

System 100 is a system for providing ultrasound images, in real time, to a consulting doctor that is in a remote location. Communication device 101 may be used by the one conducting the ultrasound examination to communicate with the consulting doctor during the examination and/or with a server for scheduling the ultrasound examination and/or reporting results of the examination.

Ultrasound machine 102 is a device for capturing ultrasound images of a patient. The ultrasound machine outputs image signals in a format appropriate for a television.

Video out 104 is the video signal produced by ultrasound machine 102, for producing an image based on the ultrasound signals produced by ultrasound machine 102.

EDID 106 is a data structure provided by the ultrasound system to describe the ultrasound system's capabilities to the transcoder (which is the video source). EDID 106 is the Extended Display Identification Data (EDID) produced by ultrasound machine 102. The ultrasound machine 102 uses an Extended Display Identification Data (EDID) conformation in order to output data in a resolution that the video transcoder can handle, which can be mitigated by an EDID emulator. For example, the EDID 106 may include a manufacturer name, serial number, product type, a phosphor or filter type, timings of a display device supported by the transcoder, image size, luminance data, and optionally a pixel mapping of the display device on which the ultrasound image will be displayed.

Optionally EDID 106 may be located in a separate device than the transcoder. EDID device 106 receives the signals produced by ultrasound machine 102 and sends image signals that are appropriate for a transcoder to a transcoder. The EDID 106 enables the ultrasound machine 102 to identify the characteristics of the ultrasound system 100 to the video transcoder.

Transcoder 108 (e.g., a Teradek video transcoder or encoder) may be an encoder, and may include a computer specifically programed (e.g., possibly hardwired) for converting video signals into WiFi signals. Transcoder 108 may include a codec that converts video signals to WiFi signals. Transcoder 108 receives the signal from video out 104 with the information entered into EDID 106 and reformats the incoming information according to the information in EDID 106. Transcoder 108 converts (or transcodes) video signals to WiFi signals by converting signals encoded for a video device, such as a television, and changes the coding to the coding used for WiFi. WiFi 110 is a wireless/radio frequency signal for a wireless local network, which may be based on the IEEE 802.11 standard, which is produced by transcoder 108 and sent to a mobile hotspot or router. In an embodiment, transcoder 108 is platform independent. The ultrasound machine 102 and transcoder 108 may be located in a mobile unit.

Mobile hotspot/router 112 is a portable device that connects to a Wide Area Network (WAN), such as the Internet and which may be used to setup a wireless network at a location, where the ultrasound machine is located. Mobile hotspot/router 112 receives the WiFi signals and sends the signals to a network. Mobile hotspot/router 112 may convert the signals from the format/coding/protocol used by WiFi system to a format/coding/protocol used by the network. Optionally, communication device 101 may be combined with mobile hotspot 112 and/or transcoder 108.

At the time of the examination, the ultrasound machine 102, video out 104, EDID 106, transcoder 108, Wifi 110, and mobile hotspot/router 112 may be located at a hospital. Alternatively, at the time of the examination, the ultrasound machine 102, video out 104, EDID 106, transcoder 108, Wifi 110, and mobile hotspot/router 112 may be located at a mobile clinic or other clinic.

Mobile unit 113 is a unit that includes ultrasound machine 102, video out 104, EDID 106, transcoder 108, and optionally mobile hotspot/router 112. Mobile unit 113 may be housing that houses ultrasound machine 102, video out 104, EDID 106, transcoder 108, and optionally mobile hotspot/router 112 so that ultrasound machine 102, video out 104, EDID 106, transcoder 108, and optionally mobile hotspot/router 112 may be relatively easily transported from location to location. While within mobile unit 113, ultrasound machine 102, video out 104, EDID 106, transcoder 108, and optionally mobile hotspot/router 112 are connected together so as to be operational and ready to use anywhere. Mobile unit 113 may be a van or may be normally located within a van that transports ultrasound machine 102, video out 104, EDID 106, transcoder 108, and optionally mobile hotspot/router 112 to different locations for capturing ultrasound images of patients.

Network 114 may be a Wide Area Network (WAN). Network 114 may be the Internet. Network 114 may be any combination of phone networks, ordinary phone network, mobile phone network, and/or computer networks, which may include the Internet. Network 114 receives the signals from the Mobile hotspot/router 112 and sends the signals to a service (e.g., a live streaming service) for streaming the content to the doctor.

Patient database 116 is a database of records having information about patients. Optionally, patient database 116 may be a relational database. The information about the patients may be retrieved from the patient database and sent along with the ultrasound images (or at another time) to the doctor. Optionally, the ultrasound images may be sent from transcoder 108, via WiFi 110 and Mobile hotspot/router 112, through network 114 to patient database 116 for storage. Alternatively, the ultrasound images may be delivered to patient database 116, when ultrasound machine 102 returns from visiting a patient.

Livestreaming service 118 receives the signals carrying the ultrasound images, via network 116, from the transcoder 108, and then delivers the content to the doctor as streaming media. In other words, the content is delivered to the doctor in a manner so that the doctor can view the contents as the contents are being received (as opposed to waiting to receive the entire content before being able to see the images). Optionally, livestreaming service 118 may be provided by a server running Real Time Messaging Protocol (RTMP) or another protocol that facilitates streaming data (e.g., to the consulting doctor). The patient's records may be automatically sent with the ultrasound images to the consulting doctor during the examination or after the examination. Using patient database 116 for storing medical records of patients is optional.

Server 120 is optional. Upon preparing a report and/or scheduling an appointment, server 120 may retrieve records from patient database 116 and send the patient data to the consulting doctor and/or ultrasound practitioner. In this specification, the word sonographer and ultrasound practitioner may be substituted one for another wherever they occur to obtain a different embodiment. In this specification, any type of doctor and the consulting doctor, may be substituted one for another wherever they are referenced to obtain a different embodiment. In this specification, any type of medical equipment and the ultrasound machine may be substituted one for another wherever they are referenced to obtain a different embodiment. In this specification, any medical professional competent in using the equipment in question and the ultrasound practitioner may be substituted one for another wherever they are referenced to obtain a different embodiment. Server 120 may store a user interface for setting up and scheduling appointments and optionally for viewing patient information. The consulting doctor may communicate by e-mail with server 120. The server 120 may send e-mails and/or other messages to the doctor regarding requests for appointments and the consulting doctor may reply, via a link in the e-mail to accept or refuse an appointment. Alternatively, the consulting doctor may login to server 120 to view requests for appointments and to approve, refuse, and/or cancel appointments. Optionally, patient database 116 and server 120 may be in the same place and/or may be the same machine. Additionally or alternatively, live streaming service 118 and transcoder 106 and/or server 120 may be in the same location and/or be the same machine.

User interface 122 may be used for setting up appointments, scheduling appointments, viewing information about upcoming appointments, and optionally for viewing patient data. A consulting doctor may use the user interface 122 to request and schedule an appointment, and/or the ultrasound practitioner may use the user interface 122 to confirm the appointment. Alternatively or additionally, the patient, care taker of the patient, or an owner of a patient (if the patient is a pet) may use the user interface 122 to request and schedule an appointment and/or the consulting doctor may use the user interface 122 to confirm the appointment. User interface 122 may send e-mails to ultrasound practitioners (or optionally to consulting doctors), requesting whether a particular time and date is good for an appointment, and receive e-mails from the ultrasound practitioner (and/or consulting doctor) confirming that a particular time and date are good for an appointment or refusing to schedule an appointment at a particular time and date.

User Interface (UI) 122 may be displayed to the medical personnel performing the ultrasound. UI 122 may include one or more tools (e.g. buttons and/or fields), which when activated request the user to input information about the patient demographics in order to locate an appropriate clinic, consulting physician, and/or practitioner for conducting the examination in the field. UI 122 may include one or more fields for entering information about why the consulting physician is requesting the examination (or why a patient needs to see the consulting physician). UI 122 may be associated with software that sends alerts to the consulting physicians and/or ultrasound practitioner about upcoming sonograms, which may include sending e-mails, text messages, calendars, updates for calendars, and/or worklists. The doctor's interpretations of the ultrasound images, diagnosis, recommendations for treatment procedure and treatment plans may also appear on the UI 122. The UI 122 may be displayed on a machine that communicates with the transcoder 108. UI 122 may be located on a remote server and/or at a computer that is connected to the transcoder 108 by a local network and/or network 114.

Optionally, another UI 122 may be provided for the consulting physician to enter the diagnosis, review the patient's records, view ultrasound images, and view any questions and/or comments that were sent by the one taking the ultrasound image and that were sent with/or accompanying the ultrasound images.

Instead of, or in addition to the UI 122, the server 120 may store a downloadable app, which may be downloaded by the consulting doctor and/or a downloadable app, which may be downloaded by the medical personnel operating the ultrasound system. Then the consulting doctor and/or the ultrasound practitioner may communicate with one another, via the webserver (e.g., server 118) using their respective apps.

App 123 is a downloadable app for communicating with the ultrasound practitioner running ultrasound machine 102, via server 120, with the consulting doctor. In the example of FIG. 1, App 123 is shown as being stored on server 120 for downloading and is shown after being downloaded, as stored on one or more of doctor devices 126a-n.

Router 124 receives signals from the network 114 and sends the signals to a device of the consulting doctor.

Consulting doctor devices 126a-n may include any of a variety of different devices that the doctor uses when communicating with a network. Consulting doctor devices 126a-n may include a smart phone, a laptop computer, a desktop computer, and/or a tablet computer, for example.

FIG. 2 shows a block diagram of another embodiment of an ultrasound system 200. In an embodiment, ultrasound system 200 includes ultrasound machine 102, video out 104, transcoder 108, WiFi 110, mobile hotspot/router 112, mobile unit 113, network 114, patient database 116, optional server 120, UI 122, app 123, router 124, and doctor devices 126a-n, which were described above in conjunction with FIG. 1. System 200 may also includes signal formatting 202 and live streaming service 204. In other embodiments ultrasound system 200 may not have all of the elements or features listed and/or may have other elements or features instead of or in addition to those listed.

System 200 differs from system 100 in that EDID 106 is not used and the livestreaming service is provided locally. For example, transcoder 108 receives the signal from ultrasound machine 102 and converts the signal to one appropriate to a video (e.g., using a different data structure or standard than EDID or by detecting the resolution and other information about the signal from ultrasound machine 102) and then live streams the ultrasound images, via hotspot/router 112 and/or network 114, to the location and/or devices of the consulting doctor.

FIG. 2 shows an alternative embodiment in which the transcoder accepts the signal from the ultrasound device directly, and the live streaming service is combined with the transcoder, so that the transcoder live streams the ultrasound image to the consulting physician.

Signal formatting 202 may be a different data structure than EDID or may be based on a different standard than EDID. Signal formatting 202 may be software that automatically detects the format of the incoming signal and optionally converts the signal and data to a format that is appropriate for transcoder 108. Live streaming service 204 is a live streaming service that is located in transcoder 108, allowing transcoder 108 to send streaming media, live to the doctor.

FIG. 3A shows a block diagram of an embodiment of an ultrasound system 300. In an embodiment, ultrasound system 300 includes ultrasound machine 102, video out 104, EDID 106, transcoder 108, Wifi 110, mobile hotspot/router 112, mobile unit 113, network 114, patient database 116, live streaming service 118, optional server 120, UI 122, app 123, router 124, and doctor devices 126a-n, which were described in conjunction with FIG. 1. Ultrasound system 300 also includes scaler 302. In other embodiments ultrasound system 300 may not have all of the elements or features listed and/or may have other elements or features instead of or in addition to those listed.

FIG. 3A shows another embodiment 300 of the ultrasound system. The ultrasound system of FIG. 3A is similar to that of FIG. 1. However, in the embodiment of FIG. 3A there is scaler that scales the resolution of the image provided by the ultrasound machine to the transcoder. The scaler of FIG. 3A is used instead of the EDID 106 (or in addition to EDID 106) of FIG. 3A. The scaler 302 of FIG. 3A (or the EDID 106 of FIG. 1) is located between the ultrasound machine 102 and the transcoder 108 (e.g., a Teredek Video encoder). Scaler 302 scales the image received from ultrasound machine 102, according to the information in EDID 106 (which is a data structure), to a scale that is appropriate for transcoder 108. Optionally, scaler 302 may be incorporated within transcoder 108 or ultrasound machine 102.

For example, if the EDID 106 does not provide the image in a resolution that the transcoder accepts, then a scaler 302 may scale the resolution to one that is accepted by the transcoder 108. In an embodiment, the EDID 106 and the scaler are only used if needed for communicating with the particular transcoder available. Although scaler 302 and transcoder 108 are shown as hardware devices in other embodiments the EDID 106, scaler 302, and/or transcoder 108 may be replaced with software running on a computer.

Referring to FIGS. 1-3A, a signal from the video out of the ultrasound machine 102 (or other High Technology Medical Instrument (HTMI)) is sent to a video transcoder 108 and forwarded to a network 114, e.g., via WiFi 110. The video feed from the ultrasound machine 102 is received from the transcoder 108 by a mobile broadband cellular router (mobile hotspot/router 112). In an embodiment, the mobile hotspot/router 112 routs information from the local network 114 to live streaming service 118, so that the information can be streamed to the consulting doctor.

In an embodiment, instead of the livestreaming service 118 (which is located remotely), ultra sound images may be sent to server 102 and sent form server 120 to one of consulting doctor devices 126a-n, using Real Time Messaging Protocol (RTMP) (or another protocol for communicating in real time). Alternatively, transcoder 108 may optionally include server software and the ultrasound images may be sent using RTMP directly to one of consulting doctors devices 126a-n, which may run software for viewing the image (e.g., a Flash player or other vector software).

RTMP is a TCP-based protocol and maintains persistent connections while allowing for low-latency communication. To deliver streams smoothly and transmit as much information as possible. RTMP splits streams into fragments, and the size of the fragments is negotiated dynamically between the client and server. Sometimes, the fragments are kept unchanged. In an embodiment, the default fragment sizes are 64 bytes for audio data, and 128 bytes for video data and most other data types. Fragments from different streams may be interleaved, and multiplexed over a single connection. With longer data chunks, the protocol may carry only a one-byte header per fragment, in order to keep the overhead small. In an embodiment, the interleaving and multiplexing is done at the packet level, with RTMP packets across several different active channels being interleaved in such a way as to ensure that each channel meets its bandwidth, latency, and other quality-of-service requirements. In an embodiment, the RTMP packets that are interleaved are treated as indivisible, and are not interleaved on the fragment level.

The RTMP may define several virtual channels on which packets may be sent and received, and which operate independently of each other. For example, there may be a channel for handling RPC requests and responses, a channel for video stream data, a channel for audio stream data, a channel for out-of-band control messages (fragment size negotiation, etc.). Optionally, during an RTMP session, several channels may be active simultaneously at any given time. When RTMP data is encoded, a packet header is generated. The packet header may specify, amongst other matters, the ID of the channel on which the packet is to be sent, a timestamp of when the packet was generated (if necessary), and the size of the packet's payload. This header may then be followed by the actual payload content of the packet, which may be fragmented according to the currently agreed-upon fragment size before the packet is sent over the connection. In an embodiment, the packet header itself may never be fragmented, and the size does not count towards the data in the packet's first fragment. In other words, in this embodiment, only the actual packet payload (the media data) is subject to fragmentation.

The Real Time Messaging Protocol (RTMP) streams audio, video, and/or other data over the Internet (or another network), between a Flash player (or other vector graphics software) and a server. In other embodiments, another vector graphics player, which may use a different protocol (other than RTMP), could be used instead of Flash. The RTMP provides a multimedia software platform for displaying the ultrasound image. Optionally, the RTMP provides the multimedia platform, with the medical records of the patient, which may be displayed with a Flash player. RTMP can capture the ultrasound images and allows streaming of the ultrasound images as still or video images, which may be accompanied by an audio track. In an embodiment, the files sent to the consulting physician are in MP4 format.

Optionally, e.g., using the Flash software (or another streaming media software that allows manipulation of text while the media is being played), the ultrasound practitioner and/or the consulting physician may be able to annotate the ultrasound images and send the images with the annotations. The above description, is just one embodiment. All or most of the components can be swapped for different models from different manufacturers. Different devices may integrate one or more of the above devices.

In another embodiment, the full system may include a front facing web app for requesting, performing, and interpreting the ultrasound data. The full system may include a database storing a standard Picture Archive and Communication System/Radiology Information System (PACS/RIS) and DVR archive associated with each examination. The PACS/RIS and DVR archive may be used to store, retrieve, distribute, analyze, and digitally process the ultrasound images taken. The full system may also include a RTMP server to handle live feeds, so that the consulting doctor may see the ultrasound images as the ultrasound images are being taken and, in real time (e.g., while receiving the live feed of the ultrasound images being taken), request the medical personnel taking the ultrasound image to take other images based on the initial images received. In an embodiment, the system of storing and retrieving records is nonproprietary. In an embodiment, in the systems of FIGS. 1-3A, the ultrasound video output sends a signal (e.g., having image data (or data of another type) to the video transcoder/uploader, which in-turn sends the data to a wireless broadband device, which forwards the signal to a RTMP Server and then the signal is sent to any computer, work station handheld device (e.g., a smart phone) where the examination can be viewed live.

The examination may be performed live in real time by an ultrasonographer. The hardware configuration of FIGS. 1-3A makes it possible for the consulting specialist (radiologist, cardiologist, internist, etc.) to observe the examination and, based on what is seen, to guide the ultrasonographer during the study. After the study is completed, the specialist has already made an interpretation and can give a diagnosis. Not only can a report be generated immediately following the ultrasound, but further, a face-to-face consultation may be held with the rDVM so the referring physician can discuss the case in a clinical context and also devise a treatment plan for the patient together.

In an embodiment, the system is coordinated with an EMR (electronic medical record) system, which may be stored in patient database 116. In an embodiment, the EMR may be standard, non-proprietary, fully featured, and freely available to all referring Doctor of Veterinary Medicines (rDVMs). The EMR may serve as the basis for all record keeping and include mechanisms for sharing, and the EMR may be fully integrated with the above described portal system. In an embodiment, the EMR may serve as a fully integrated system through which any doctor can submit a case to be reviewed for consultation with a specialist of choice or with a preselected default specialist. In an embodiment, the consulting doctor (e.g., a specialist of any and all disciplines) is given access to all patient information, with pertinent information first, such as the most recent diagnostics.

FIG. 3B shows a representation of an embodiment of an environment 330 in which the ultrasound system of FIGS. 1-3A may be used. Environment 330 may include mobile clinic 332, clinic 334, and residential home 336. In other embodiments, environment 330 may include additional components and/or may not include all of the components listed above.

Mobile clinic 332 may be a van or other vehicle, which may be equipped with mobile unit 113. In an embodiment, a referring Doctor of Veterinary Medicines (rDVMs) (or general practitioner) requests an examination for an ultrasound. Then, a consulting physician receives the request, and the ultrasound team goes to the rDVM's clinic to perform the examination.

Mobile unit 113 may be moved from mobile clinic 332 into hospital 334 for conducting the examination. While at clinic 334, transcoder 108 may communicate with the router already installed in clinic 334, or may use optional mobile hot spot/router 112 that optionally may be stored within mobile unit 113.

Residential home 336 may be a home of the patient. Optionally, mobile clinic 332 may be capable of traveling to residential home 336 and conducting an examination of the patient there. Optionally, mobile clinic 332 may include a table and/or other medical equipment for conducting the examination.

FIG. 4 shows a block diagram of an ultrasound system 400 used in the system of FIG. 1, 2, or 3A. The ultrasound system may include output system 402, input system 404, memory system 406, EDID 408, processor system 410, and transducer system 412. In other embodiments, ultrasound system 400 may include additional components and/or may not include all of the components listed above.

Ultrasound system 400 is an example of an ultrasound system that may be used for the ultrasound machine of FIG. 1, 2, or 3A.

Output system 402 may include any one of, some of, any combination of, or all of a monitor system, a handheld display system, a printer system, a speaker system, a connection or interface system to a sound system, an interface system to peripheral devices and/or a connection and/or interface system to a computer system, intranet, and/or internet, for example.

Input system 404 may include any one of, some of, any combination of, or all of a keyboard system, a mouse system, a track ball system, a track pad system, buttons on a handheld system, a scanner system, a microphone system, a connection to a sound system, and/or a connection and/or interface system to a computer system, intranet, and/or internet (e.g., IrDA, USB), or Bluetooth, for example.

Memory system 406 may include, for example, any one of, some of, any combination of, or all of a long term storage system, such as a hard drive; a short term storage system, such as random access memory; a removable storage system, such as a floppy drive or a removable drive; and/or flash memory. Memory system 406 may include one or more machine-readable mediums that may store a variety of different types of information. The term machine-readable medium is used to refer to any non-transient medium capable carrying information that is readable by a machine. One example of a machine-readable medium is a non-transient computer-readable medium. Another example of a machine-readable medium is paper having holes that are detected that trigger different mechanical, electrical, and/or logic responses. Memory 406 may store the EDID 106 of FIG. 1, 2, or 3A.

Processor system 410 may include any one of, some of, any combination of, or all of multiple parallel processors, a single processor, a system of processors having one or more central processors and/or one or more specialized processors dedicated to specific tasks. Processor system 410 receives input from input system 404 and sends output to output system 402. Processor system 410 retrieves machine instructions from memory system 406, and implements the machine instructions. Processor 408 may store ultrasound images in memory system 406 and causes ultrasound images to be displayed on a monitor of output system 402 and/or to be sent to another device for live streaming, via output system 402. Processor system 410 may format the ultrasound images based on information stored in the EDID 106 prior to sending the ultrasound images in the transcoder.

Transducer 42 transduces electrical signals into ultrasound signals and/or ultrasound signals into electrical signals. Transducer 412 captures ultrasound images under the control of processor 410.

FIG. 5 shows a block diagram of a server system 500 used in the system of FIG. 1. The server system may include output system 502, input system 504, memory system 506, UI 508, server module 510, optional RTMP 512 and processor system 514. In other embodiments, server system 500 may include additional components and/or may not include all of the components listed above.

Output system 502, input system 504, memory system 506, UI 508, server module 510, optional RTMP 512, and processor system 514 may be similar to output system 402, input system 404, memory system 406, and processor system 410. However, unlike memory system 406, memory system 506 stores a UI, a server module and optionally stores the RTMP, which may be implemented by processor 514. Optionally RTMP may be located on a different machine and/or at a different location that server system 500.

Optionally, transcoder 108 may have the same structure as server system 500, but instead of or in addition to memory 506 storying RTMP, memory 506 may store a codec for translating video signals into WiFi signals. Similarly, patient database may use the hardware of FIG. 5, but memory system 506 stores a database having the patient information. Optionally, the client database 116 and live streaming service 118 may have the same structure as server system 500, but patient database 116 may store in memory 506 a database that holds information about patients. Optionally, there may be a separate computer between transcode 108 and router 112 that communicates via router 124 with one of referring doctor devices 126a-n, which may have the same structure as server 500.

FIGS. 6A and B shows an example of a form 600 for requesting an examination. Form 600 may include hospital information 602 (FIG. 6A), case information 604 (FIG. 6A), Pertinent history 606 (FIG. 6A), service requested 608 (FIG. 6B), practitioner comments 610 and submit request 612 (FIG. 6B). In other embodiments, form 600 may include additional components and/or may not include all of the components listed above.

FIGS. 6A and 6B are two parts of the same page. FIG. 6A shows the upper portion and most of the form 600, whereas FIG. 6B shows the bottom of form 600.

Form 600 may include icons to aid in determining the information requested, such as an icon to indicate the patient (e.g., a paw), an icon to indicate the client, an icon to indicate a hospital/clinic.

Hospital information 602 includes fields for entering the patient name, the referring doctor, the e-mail address where the report should be sent, the e-mail address of those that should receive copies of the report (a cc list). Optionally, the patient identifier, sex, age, breed and type of animal, the owner of the animal, the contact information of the owner, and/or other information related to the patient may be automatically retrieved from the patient database 116 and included in page 600 and/or fields not yet filled in on form 600, based on the name of the pet and/or owner or other information entered into form 600.

Hospital information 602 includes details about the hospital where the ultrasound examination will take place. For example, the hospital information may include the name of the hospital, the referring physician, the telephone number of the referring physician, the e-mail address of the referring physician, a list of those that should be cc'ed on communications relating to this appointment. Hospital information may include other contact information and/or information about the hospital, such as the location and contact information of the hospital. The hospital information may be replaced with information about a clinic or other location where an ultrasound examination is requested to be performed. In other embodiments, the hospital information may be replaced with clinic information or information about the location where the examination will take place.

Case information 604 includes information about the case, such as information about the patient. If the patient is a pet, the information may include the name of the client or owner of the pet, the name of the pet, the patient identifier (e.g., a patient identification number), the type of animal (e.g., dog, cat, lizard, eagle, etc.), the breed of the pet, the sex of the patient, the age of the patient, the weight of the patient, the case number, and the preferred date of the examination.

Pertinent history 606 includes a text field in which the requestor can enter text describing pertinent history. Service requested 608 includes a list of possible services with check boxes, so that the requestor can select a service to request by just checking a box. Service requested 608 may include a field that has a description of the area of the body being examined and/or a description of the test or examination being performed. In the example of FIGS. 6A and 6B, the service requested is an ultrasound of the abdomen.

Practitioner comments 610 includes a text field in which the requestor can add any other special concerns or requests related to this examination and/or patient.

Submit button 612 is selected by the consulting doctor when the consulting doctor has filled out form 600. Selecting submit button 612 may cause a confirmation to be sent to the consulting doctor confirming that the request for the appointment has been received and may cause a request to be sent to the ultrasound practitioner, requesting the appointment.

FIG. 7 shows a screenshot of an example of a page 700 for acknowledging a request for an ultrasound examination. Page 700 may include a body 702, hospital information 704, case information 706, or service requested 708. In other embodiments, page 700 may include additional components and/or may not include all of the components listed above.

Page 700 may be sent to a consulting doctor that requested an ultrasound examination (e.g., via form 600) from an ultrasound providing service. Page 700 may be sent as an e-mail or may appear after filling out a form at a website or in an app.

Body 702 is the body of page 700, which may include a statement acknowledging the receipt of the request for an ultrasound image or examination, and may inform the requestor that another communication will follow, regarding whether an ultrasound examination can be performed at the requested time. Body 702 may include the date and time requested and a contact number of the provider of the ultrasound examination.

Hospital information 704, case information 706, service requested 708 (of FIG. 7) may be the similar to, hospital information 602, case information 604, and service requested 608 (of FIGS. 6A and 6B) respectively. However, hospital information 602, case information 604, and service requested 606 included fields for the requestor of the service to fill in, whereas page 700 is sent to the requestor as a confirmation that the information received is the same as the information that the requestor intended to enter.

Specifically, hospital information 704 includes details about the hospital (or clinic or other location) where the ultrasound examination will take place, similar to hospital information 602. Case information 706 includes information about the case, such as information about the patient, similar to case information 604. Service requested 708 may include a field that has a description of the area of the body being examined and/or a description of the test or examination being performed. In the example of FIG. 7, the service requested is an ultrasound of the abdomen.

FIG. 8 shows a screenshot of an example of a page 800 that the ultrasound practitioner performing the examination may use to respond to the request for an appointment or an ultrasound examination. Page 800 may include a header 802, a confirm button 804, a reschedule button 806, and/or a send report button 808. Additionally, page 800 may include hospital information 812, case information 814, and/or service requested 816. In other embodiments, page 800 may include additional components and/or may not include all of the components listed above.

The page 800 of FIG. 8 is displayed in response to the user selecting the submit button 704 of FIG. 7 or in response to the practitioner sending an ultrasound image (or other medical image or test result) to the consulting physician. The consulting physician may select to confirm the appointment, reschedule the appointment, or send a report. The pages of FIGS. 6A, 6B, 7 and 8 may be pages of an app that is down loaded to the user's smart phone or another user machine. Form 600, page 700, and page 800 may be available as a Short Message Service (SMS) page, and/or may be viewable, via a browser on a computer or smart phone.

Page 800 may be sent to a practitioner from whom ultrasound service is being requested. Similar to page 700, page 800 may be sent as an e-mail or may be presented to the practitioner after the practitioner logs in to the system.

Optionally, when any of confirm button 804, reschedule button 806, or report button 808 are pressed a webpage or page of an app is opened for performing a related task and/or verifying that the desired task was performed.

Confirm button 804 may be pressed by the ultrasound practitioner to confirm the appointment. Pressing on confirm button 804 may automatically open a browser window of a webpage or a page of a web app indicating that the appointment is confirmed and a calendar may be automatically updated to show that the practitioner has an appointment at the requested time.

Reschedule button 806, when pressed may cause a page of an app or a website to open that has fields that the user can fill in with information for requesting a different date and/or time for the requested examination and/or for indicating times and dates that are still open or times and dates that are not available for an appointment. After pressing reschedule button 806, a message may automatically be sent to the requestor of the appointment, requesting the requestor to reschedule the appointment and optionally including information about what time slots are still open.

Send report button 808, upon being pressed, may cause a message to be sent to the requesting doctor and/or other interested parties. The message may include the sonogram and information about the patient, such as an identifier of the patient and the test performed.

Hospital information 812, case information 814, and service requested 816 (of FIG. 8) may be the same as hospital information 602 or 704, case information 604 or 706, service requested 606 or 708 (of FIG. 7), respectively. However, the information provided on page 700 of FIG. 7, allows the requestor (e.g., the referring physician) to confirm that information about the requested appointment, the examination requested, and patient entered are what the requestor intended, whereas the information provided in page 800, FIG. 8, informs the practitioner of the information of the examination that is being requested so that the practitioner can determine whether to accept the appointment. In an embodiment, pages 600, 700 and 800 may be in clear lucid English or another language.

FIGS. 9 and 10 together form a method of use of the ultrasound system 100. FIG. 9 relates to use of an application that may be used with the method of use of the rest of the ultrasound system 100. FIG. 9 is an example of a method performed by a server, which is therefore from the point of view of the server 120 (or another server). The consulting doctor and ultrasound practitioner operating the ultrasound machine 102 implement methods on their respective communication devices (communication device 101 and one of consulting doctor devices 126a-n) that are complementary to that of FIG. 9.

In step 902, a request for an examination is received by server 120 from the doctor, via one of consulting doctor devices 126a-n. The request may be received, via e-mail, filling out a form on a website, or filling in fields in an application on a smart phone, tablet, or other computing device, and selecting a submit, enter, or send button, for example. The request may include information about the patient, the hospital where the examination will occur, a time and date for the examination, and procedure to be performed and/or region that needs to be examined. The consulting doctor may use form 600 of FIGS. 6A and 6B (or another form) to send the request of step 902.

In step 904, in response to step 902, a confirmation that the request for the appointment was received is sent from server 120 back to one of the consulting doctor devices 126a-n. As part of step 904, the form 700 of FIG. 7 (or another form) may be sent to one of the consulting doctor's devices 126a-n step 902.

In step 906, which may also be in response to receiving the request for the appointment in step 902, the request is sent to, presented to, or viewed by the ultrasound practitioner. The ultrasound practitioner may login to server 102 to check for incoming requests for appointments (as well as possibly check for other purposes, such as viewing other communications related to examinations already performed or patient information). The request may include similar information as was in the acknowledgement of the request sent to one of the devices of the consulting doctor. As part of step 906, page 800 of FIG. 8 may be sent to the device of the practitioner, e.g., communication device 101.

In step 908, a reply is received at server 120 from the ultrasound practitioner. The reply may result from the ultrasound practitioner selecting one of confirm button 804 or reschedule button 806.

In step 910, a determination is made whether the appointment was accepted. If the appointment was not accepted (e.g., server 120 detects that reschedule button 806 was selected), the method proceeds to step 911.

In step 911, a message is sent, presented, or made available to the consulting doctor that the appointment cannot be scheduled at that time, and optionally a request to reschedule the appointment is sent to one of consulting physician devices 126a-n. After step 911, the method returns to step 902 and waits to receive a new or revised request for an examination, which may be the same as the prior request, except with a different time and/or day being requested for the examination.

Returning to step 910, if the appointment is accepted (e.g., if server 120 detects that button 804 was selected), the method proceeds to step 912.

In step 912, a calendar at server 120, communication device 101, and/or one of consulting doctor devices 126a-n is updated to show that an appointment has been scheduled during the time slot indicated in the request.

In step 914, a confirmation of the appointment is sent to communication device 101 and/or one of consulting doctor device's 126a-n, confirming that the appointment was scheduled. Optionally, one or more reminders may be sent reminding the ultrasound practitioner and/or the consulting doctor about the appointment. After step 914, method 1000 of FIG. 10 is performed (which will be discussed below in conjunction with FIG. 10).

Next, in step 916 (e.g., after the ultrasound examination is performed), a report of the examination is sent from the ultrasound practitioner (optionally, via server 120) to one of consulting doctor devices 126a-n, which may be accomplished by the ultrasound practitioner selecting send report button 808. The report may be sent directly to one of consulting doctor devices 126a-n and/or one of consulting doctor devices 126a-n or may be sent via server 120. For example, as part of step 916, for example, as a result of selecting send report button 808, the report may also be sent to patient database 116, and a record containing information about the patient is updated to include information about the examination, such as the ultrasound images and comments added by the ultrasound practitioner. For example, as result of pressing report button 808, a message may be sent from communication device 101 to server 120, which retrieves the ultrasound images and then sends the images to patient database 116. Alternatively, selecting report button 808 may send a message directly to transcoder 108, which causes the images to be sent to the patient database 116 and/or one of consulting doctor devices 126a-n. In an embodiment, each of the steps of method 900 is a distinct step. In another embodiment, although depicted as distinct steps in FIG. 9, steps 902-916 may not be distinct steps. In other embodiments, method 900 may not have all of the above steps and/or may have other steps in addition to or instead of those listed above. The steps of method 900 may be performed in another order. Subsets of the steps listed above as part of method 900 may be used to form their own method.

FIG. 10 is an example of a method of performing an ultrasound examination, which may be performed after step 914 and before step 916 of FIG. 9, for example. In step 1002 the ultrasound images are recaptured by ultrasound machine 102. In step 1004, the images are sent from ultrasound machine 102 to transcoder 108. In step 1004, the signals leave ultrasound machine 102, via video out 104.

In step 1006, the images are rescaled for ultrasound machine 101. Optionally, prior to reaching transcoder 108, the images may pass through a scaler 302, where the images are rescaled for transcoder 108. The ultrasound images are rescaled to a resolution accepted by transcoder 108, based on the information stored in EDID 106 (FIG. 1 or 3A) about the images coming from transcoder 108. Alternatively, signal formatting 202 may rescale the images for the transcoder 108.

In step 1008, transcoder 108 transcodes the signals to WiFi signals 110 and sends the WiFi signals 110 to mobile hotspot/router 112. In step 1010, if mobile hotspot/router 112 is a router connected directly to the Internet or other WAN that uses an IP protocol, then the mobile hotspot/router converts the WiFi signals to IP protocol signals, which are sent into the WAN. If mobile hotspot/router 112 is a mobile hotspot, the WiFi signals are converted to mobile network signals (e.g., using the protocol of a mobile phone network) and a server or router within the mobile network converts the mobile network signals to IP protocol signals. On step 1012, whether mobile hotspot/router 112 is a mobile hotspot or a router, mobile hotspot/router 112 automatically causes the images to be sent to livestreaming service 118 (optionally live streaming service 118 may be located within server 120). In step 1014, the ultrasound images are streamed to one of consulting doctor devices 126a-n. The images may be streamed to one of consulting doctor devices 126a-n, while the patient is still in the examination facility, optionally as the images are being captured, so that the doctor can ask for more images based on the images being received. One of consulting doctor devices 126a-n may be in contact with practitioner at the examination as the images are being taken, so that the consulting doctor can communicate with the ultrasound practitioner as the ultrasound images are being captured.

In an alternative embodiment, transcoder 108 may include a live streaming service to stream the images to one of the consulting doctor device 126a-n, instead of sending the signal to livestreaming service 118. In an embodiment, each of the steps of method 1000 is a distinct step. In another embodiment, although depicted as distinct steps in FIG. 10, step 1002-1014 may not be distinct steps. In other embodiments, method 1000 may not have all of the above steps and/or may have other steps in addition to or instead of those listed above. The steps of method 1000 may be performed in another order. Subsets of the steps listed above as part of method 1000 may be used to form their own method.

### Pseudocode Snippets

Below are pseudocode snippets that implement various functions of the application. The pseudocode snippets below may be stored in memory system 506 of server 500 or memory system 406 of server 500.

Service Request pseudo code snippet

// An example of one possible embodiment of creating a Service Request.

class ServiceRequestsController <ApplicationController

private

Define Service_Request_Parameters a Service Request Object can Receive

parameters.require(:service_request).permit(
:hospital_name,
:telephone_number,
:email_address,
:email_cc,
:referring_doctor,
:client_name,
:patient_name,
:patient_id,
:breed,
:patient_sex,
:patient_status,
:age,
:patient_age_unit,
:patient_weight_lbs,
:patient_weight_kg,
:case_number,
:preferred_exam_date_and_time,
:pertinent_history,
:study_abdomen,
:study_cardiac,
:study_re_evaluation,
:study_thorax,
:study_musculoskeletal,
:study_effusion_check,
:study_two_cavities,
:study_neck,
:study_abbreviated_1_cavity,
:study_cystocentesis,
:study_thoracocentesis,
:study_abdominal_centesis,
:study_general_centesis,
:study_guided_aspirate,
:study_guided_biopsy,
:study_urogenital,
:study_traumatic_catheterization,
:practitioner_comments_requests)

Create a New, Empty Service Request Object and Check for Errors

@service_request = ServiceRequest.new
flash[:danger] = ALERT_MESSAGE if (ALERT_MESSAGE)

Populate Service Request Object Fields with the Values Entered by User

@service_request = ServiceRequest.new(request_parameters)

Format DateTime Before Saving Service Request

request_parameters = service_request_parameters
(request_parameters[:preferred_exam_date_and_time] != ″)
request_parameters[:preferred_exam_date_and_time] =
DateTime.strptime
(request_parameters[:preferred_exam_date_and_time],
‘%m/%d/%Y %l:%M %P’)
.to_formatted_s(:db)

Save Service Request to Database or Other Data Storage

if @service_request._save
appointmentRecords = Record.create([{ServiceRequestID:
service_request.preferred_exam_date_and_time.strftime(‘%m/%d/%Y %l:%M
%P’) + service_request.hospital_name + service_request.patient_id},
{description: service_request.case_number+
service_request.client_name + service_request.patient_name}])

Call Mailer Service to Create a Confirmation of Successful Service Request

PulseMailer.service_request_email

If the Service Request is from a New User, Enter User as a Contact

```
            Define contact_parameters
            parameters.require(
            :contact
            ).permit(
            :name,
            :email_address,
            :message)
@contact = Contact.new(contact_parameters)
```

The pseudo code above for entering a contact is optional and the rest of the pseudo code can function without the pseudo code above.

Generate Confirmation E-Mail for Service Provider

class PulseMailer < ApplicationMailer
define service_request_email(service_request)
@service_request = service_request mail(
to: ‘person1@pulsevet.net; person2@pulsevet.net;
person3@pulsevet.net; person4@pulsevet.net,
subject: “Service request from #{@service_request.hospital_
name}”)

Generate Confirmation E-Mail for User

```
define service_request_confirmation(service_request)
@service_request = service_request
              mail(
              from: ‘person1@pulsevet.net’,
              to: @service_request.email_address,
              cc: @service_request.email_cc,
              subject: ‘Service request confirmation from
Pulse Vet Imaging’)
```

Send Confirmation E-Mails to User and Service Provider

(@service_request).deliver_now
PulseMailer.service_request_confirmation
(@service_request).deliver_now
flash[:success] = “Your request has been sent, and a confirmation email has been sent to
#{@service_requestemail_address}

When a Message is Sent Via a Contact Us Form

```
Define contact_email(contact)
              if @contact.valid?
PulseMailer.contact_email(@contact).deliver_now
flash[:success] = “Your message has been sent - someone will get back to you shortly.”
@contact = contact
              mail(from: @contact.email_address, to: ‘person1@pulsevet.net;
person2@pulsevet.net; person3@pulsevet.net; person4@pulsevet.net’,
subject: “Message from #{@contact.name}”)
```

The pseudo code above for sending a message via a contact form is optional and the rest of the pseudo code can function without the pseudo code above.

### Database or Data Object Structure

In one possible embodiment, the database may be modeled as shown below. A data object capable of storing the same data may be used instead of, or in addition to, the data structure shown below.

Define Database/Data Object Schema

create_table “service_requests”,
t.string “hospital_name”
t.string “telephone_number”
t.string “email_address”
t.string “referring_doctor”
t.string “client_name”
t.string “patient_name”
t.string “breed”
t.string “sex_status”
t.integer “age”
t.string “case_number”
t.text “pertinent_history”
t.boolean “study_abdomen”
t.boolean “study_cardiac”
t.boolean “study_re_evaluation”
t.boolean “study_thorax”
t.boolean “study_musculoskeletal”
t.boolean “study_effusion_check”
t.boolean “study_two_cavities”
t.boolean “study_neck”
t.boolean “study_abbreviated_1_cavity”
t.boolean “study_cystocentesis”
t.boolean “study_thoracocentesis”
t.boolean “study_abdominal_centesis”
t.boolean “study_general_centesis”
t.boolean “study_guided_aspirate”
t.boolean “study_guided_biopsy”
t.boolean “study_urogenital”
t.boolean “study_traumatic_catheterization”
t.text “practitioner_comments_requests”
t.datetime “created_at”, null: false
t.datetime “updated_at”, null: false
t.string “email_cc”
t.string “patient_id”
t.datetime “preferred_exam_date_and_time”
t. decimal “patient_weight_lbs”
t. decimal “patient_weight_kg”
t.string “patient_sex”
t.string “patient_status”
t.string “patient_age_unit”

The above pseudo code snippet is just one example of one possible coding for defining, creating and storing Service Requests, and one possible schema for storing data.

Optionally, the database may be a relational database, which may further include a table for storing client information, a table for storing patient information, a table for storing clinic (e.g., hospital) information and a table for storing information about the referring doctor. The table for storing client information may include columns for the name of the client, contact information of the client, a list of patients owned by or cared for by the client. The table for storing patient information may include columns for the name of the patient, the care giver or owner of the patient, of the client, the type of animal of the patient, the breed of the patient, the gender of the patient and the medical history of the patient. The table for storing information about the clinic (e.g., hospital) information may include a column for the name of the clinic, the type of institution, the address of the clinic, phone number of the clinic, the e-mail address of the clinic, equipment available at the clinic, information about the network connection available at the clinic and a contact information for a contact person at the clinic. The table for storing information about the referring doctor, may include columns for contact information for the referring doctor, a list of patients of the referring doctor, the specialty of the referring doctor, if any, and notes about the referring doctor.

FIG. 11 illustrates an example of a first embodiment of a schema 1100 in which all of the information for the system (e.g., the patient information, doctor information, clinic information, client information, and patient information) are stored in one table of examination/studies performed/requests for examinations.

FIG. 12 is another embodiment of a schema 1200 in which multiple tables are used. Schema 1200 may include a separate table for each of client information, patient information, referring doctor information, clinic information, study information, and case information.

The primary key for the client information is the client id or client name, which may be a foreign key in the study table. The primary key for the patient information is the patient id, which may be a foreign key in the client table and the case table. The primary key for the clinic information is the clinic name or clinic id, which may be a foreign key in the study table. When one wants to see a combination of the information about a client with one or more of the client's pet/patients, the client and patient tables are joined using the patient id for joining the pertinent information in the tables. The status column and date of study column in the study table may be used to differentiate between studies that were completed and studies for which the referring doctor has requested a date, but the date has not yet been confirmed, such as by having statuses, such as “requested date,” “scheduled/confirmed date,” “in progress,” and “complete.” The study table is a join table, which acts as a master table that can be used to join information from any combination of the other tables together to produce different types of reports or useful information. Also, the information in all of the tables may be joined together to produce a request view having the information and columns of FIG. 11. The case table has the same primary key as the study table, which is the case number. The case table is optional and is just an extension of the study table and contains more details about the studies being requested, scheduled, in progress, and/or already performed. How much of the information about the study and which pieces of information about the study are stored in the case table instead of the study table is arbitrary. For example, the study table may just have the case number and all of the rest of the details of the study could be put in the case table or alternatively all of the case information could be stored in the study table and the case table could be removed from the schema. In each of the client, patient, referring doctor, and clinic tables there may be two separate columns (in addition to the other columns) in which one of the two columns is for an identifier of the client, patient, referring doctor, and/or clinic and the other of the two columns is for the corresponding name, respectively. Alternatively, if the number of client, patient, referring doctor, and/or clinics is small enough, the name may be used as the identifier (and so only one column for the name and identifier is used), respectively. In the case table, the column for the ultrasound images is optional. The column for the ultrasound images stores the actual ultrasound images and is left blank until the ultrasound exam is in progress or completed.

### ALTERNATIVES AND EXTENSIONS

Although the specification references livestreaming ultrasound images to a remotely located consulting doctor, ultrasound is just one example. Any medical image or medical examination may be substituted, and any practitioner competent to operate the medical examination machine may be substituted for the ultrasound practitioner. For example, the ultrasound machine may be substituted with an x-ray machine, Mill machine, thermal imaging machine, ordinary photos or videos (e.g., of wounds or other ailments), tomography machine, echocardiogram machine, elastography machine, tactile imaging machine, or positron emission tomography. Although the specification refers to veterinarians, any type of doctor may be substituted for the consulting doctor.

Although in the example above, the doctor initially requests the appointment and the ultrasound practitioner (or practitioner operating another type of medical equipment) accepts the request or the request for the appointment to be rescheduled, alternatively or additionally, the ultrasound practitioner (or practitioner operating another type of medical equipment) may initially request the appointment and the consulting doctor accepts the request or request for the appointment to be rescheduled.

Each embodiment disclosed herein may be used or otherwise combined with any of the other embodiments disclosed. Any element of any embodiment may be used in any embodiment.

Although the invention has been described with reference to specific embodiments, it will be understood by those skilled in the art that various changes may be made and equivalents may be substituted for elements thereof without departing from the true spirit and scope of the invention. In addition, modifications may be made without departing from the essential teachings of the invention.

1. A system comprising:
an ultrasound transducer for capturing ultrasound images;
a encoder for converting images from the ultrasound transducer to a format for transmission, an output of the ultrasound transducer being connected to the encoder, so that images captured by the ultrasound transducer are sent to the encoder; and
a module communicatively coupled to the transducer routing the ultrasound images to a livestreaming service.

2. The system of claim 1, further comprising a memory system associated with the encoder, the memory system including nontransient memory, the memory system storing a data structure including information about the signal produced by the ultrasound transducer to assist the encoder in handling the signal from the ultrasound transducer.

3. The system of claim 2, the data structure storing information related to the resolution of images produced by the signal from the transducer.

4. The system of claim 1 further comprising a database of patient records.

5. The system of claim 1 further comprising a router for sending signals from the transcoder to the livestreaming service.

6. The system of claim 1 further comprising a mobile hotspot for sending signals from the transcoder to the livestreaming service.

7. The system of claim 1 further comprising a server that includes a memory system storing a downloadable application for scheduling appointments ultrasound appointments that are live streamed.

8. The system of claim 1, the downloadable application including code for creating a button, which when activated confirms an appointment.

9. The system of claim 1, the downloadable application including code for creating a button, which when activated causes a request to be sent, the request requesting that an appointment be rescheduled.

10. The system of claim 1, the downloadable application including code for creating a button, which when activated causes an initiation of creation of a report related to an examination that occurred at an appointment.

11. A system comprising:
an ultrasound transducer for capturing ultrasound images; and
an encoder for converting images from the ultrasound transducer to a format for transmission, an output of the ultrasound transducer being connected to the encoder, so that images captured by the ultrasound transducer are sent to the encoder, the encoder including at least
a memory system, and
a codec,
the memory system storing at least
a scaler for scaling a resolution of an image from the ultrasound transducer to a resolution of images of the transcoder, and
a livestreaming service for streaming the ultrasound image to a location that is remote from the ultrasound transducer.

12. A method comprising:
capturing one or more ultrasound images with an ultrasound transducer;
automatically, by a machine, sending the one or more ultrasound images to a livestreaming service, the machine including a processor system;
sending a livestream of the one or more ultrasound images to a machine of a consulting physician.

13. The method of claim 12 further comprising:
receiving, at a machine, feedback from the consulting physician, prior to a practitioner that was present during the capturing of the ultrasound images leaving an examination facility where the ultrasound images were captured.
